(19) United States
(12) Patent Application Publication
(10) Pub. No.: US 2011/0082548 A1
(43) Pub. Date: Apr. 7, 2011

(54) LOW FRICTION RESURFACING IMPLANT

(75) Inventors: Robert Assell, St. Paul, MN (US); Brian P. Beaubien, St. Paul, MN (US); David Stassen, Edina, MN (US)

(73) Assignee: ZYGA TECHNOLOGY, INC., Minneapolis, MN (US)

(21) Appl. No.: 12/898,285

(22) Filed: Oct. 5, 2010

Related U.S. Application Data
(60) Provisional application No. 61/249,447, filed on Oct. 7, 2009.

(51) Int. Cl. A61F 2/08

(52) U.S. Cl. 623/14.12

(57) ABSTRACT

A low friction resurfacing implant system including a first implant component and a second implant component. The first implant component has a first bearing surface. The second implant component has a second bearing surface. The first implant component and the second implant component are each fabricated from a durable material that possesses a low coefficient of friction.

### REFERENCE TO RELATED APPLICATION

This application claims priority to U.S. Provisional Application No. 61/249,447, which was filed on Oct. 7, 2009, the contents of which are incorporated herein by reference.

### FIELD OF THE INVENTION

The present invention relates to orthopedic medicine, and more specifically to minimally-invasive tissue sparing implantable prostheses, associated tools, and methods for the resurfacing of articulating joints.

### BACKGROUND OF THE INVENTION

The hip, knee, ankle and intervertebral discs of the spine are considered load-bearing joints, while the fingers and toes are considered non-weight bearing joints. The hip, knee, and ankle are further categorized as synovial joints, while the intervertebral discs are cartilaginous joints. These joints, especially the weight bearing joints, can undergo degenerative changes due to disease, age, trauma, repetitive loading and/or genetics.

For synovial joints, these degenerative changes come in the form of arthritis, or inflammation of the joint, leading to damage of the articular cartilage. Osteoarthritis mainly damages the joint cartilage, but there is often some inflammation as well. Rheumatoid arthritis is mainly inflammatory, and can eventually destroy the joint cartilage and adjacent bone. Fracture and other forms of trauma such as from sports injuries may also lead to degenerative changes.

Osteonecrosis is a condition in which either the bone of the femoral head or femoral condyles dies. The dead bone cannot withstand the stresses of walking and as a consequence, the femoral head or condyles then collapse, become irregular in shape, and cause pain in the hip or knee joints.

Osteoarthritis (OA) is the most common of the rheumatologic musculoskeletal disorders affecting about 21-26 million of the US adult population with the knee accounting for about 6.5 million of these cases.

Once the articular cartilage becomes deteriorated from OA, the result is bone rubbing against bone. The bone-on-bone friction causes discomfort ranging from feelings of stiffness to debilitating pain and eventual loss of motion.

Treatments for OA of the knee include conservative or non-pharmacological therapy, like physiotherapy, weight management and exercise; and more generally, intra-articular injections, arthroscopic surgery and knee replacement surgery. Whereas total or partial knee replacement surgery is considered an end-of-line intervention, the less invasive surgical procedures of lavage or debridement may be recommended for earlier and more severe disease.

Both arthroscopic lavage and debridement have been performed in patients with knee joint pain, with or without mechanical problems, refractory to medical therapy.

However arthroscopic lavage and debridement for osteoarthritis of the knee is still considered experimental and investigational by insurance companies because its effectiveness has not been established.

At this time, options that help to completely relieve severe osteoarthritis, include joint replacement or fusion. As examples, approximately 200,000 total knee joint and over 300,000 hip joint replacement operations are performed annually, and typically these artificial joints only last about 10-15 years.

Progression through the clinical pathway, however, is not linear, with treatment dependent on factors such as disease severity, patient preference, medical insurance reimbursement issues, and even the medical specialty of the physician the patient sees. In addition, some patients prefer not to have invasive surgery such as knee replacement; instead, they would prefer the less invasive injections and/or arthroscopic procedures.

It is therefore the object of the invention to provide a knee resurfacing implant, system, and method for treating patients experiencing moderate to severe OA knee pain who are either too young or too old to be candidates for total knee replacement surgery.

More particularly, the present invention relates to implantable systems, and corresponding insertion methods and procedures, which provide resurfacing of the knee joint anatomy on a minimally-invasive basis without bone resection and minimal native tissue disruption to reduce or eliminate joint pain and reestablish or maintain normal or near-normal joint stabilization and motion.

### SUMMARY OF THE INVENTION

An implantable prosthesis for resurfacing weight bearing joints is provided. The prosthesis may have two or more surfaces that articulate against one another that are formed of PEEK or similar materials to provide conformability, reduced friction and improve wear capabilities while maintaining sufficient strength to operate in a weight bearing capacity.

One embodiment of the implant is a two-component system placed in the lateral and/or medial compartment of the knee between the tibial plateau and femoral condyle by means of minimally invasive surgery.

In another embodiment of the implant, the two-component system is placed between the patella and the femur.

Embodiments disclosed may include mechanical fasteners such as spikes, teeth, screws, tabs, flats folds and/or adhesives to secure the implant.

The instruments provided with the implants are intended to aid in the preparation of the implant site and implant placement. Instruments are designed for minimally-invasive delivery of implant prostheses without need for bone resection and with minimal tissue removal or disruption.

The implant is designed to help relieve pain by restoring the low coefficient of friction and durability of the articulating surfaces of the knee joint. The implant component geometry improves knee joint spacing. The articulating surfaces of the implant components are smooth and intended to mimic the lubricious surface previously provided by the healthy articular cartilage.

The implant may be conformable to the articular cartilage surfaces to the extent of restoring the original geometry of the tibial plateau and femoral condyle or the patellar and femoral articulating surfaces prior to the diseased or injured state without being so conformable to reproduce the irregularities created by the degenerated articular cartilage originally causing the pain.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a simplified view of the anatomy of the human knee joint illustrating the area in which the systems and methods of the present disclosure are useful in treating.

FIG. 2 is a simplified view of a healthy vs. osteoarthritic bone, cartilage and meniscus of the knee.

FIG. 3 is a detailed view of osteoarthritis of the knee.

FIG. 4 is a preferred embodiment of the invention indicating one half of the lateral compartment two-component implant and one half of the medial compartment two-component implant.

FIG. 5 is a reverse side of each half of the lateral and medial implants shown in FIG. 4 illustrating tissue engaging spikes.

FIG. 6 is a cross-section indicating contour of articulating surfaces and spikes of FIGS. 4 and 5.

FIG. 7 is a perspective view of implants having cap feature with side holes.

FIG. 8 is a perspective view of implants having tab feature.

FIG. 9 is a perspective view of implants having top center hole feature.

FIG. 10 is a perspective view of implants having top dual hole feature.

FIG. 11 is a side view of implanted components in knee joint

FIG. 12 is a front view of implanted components in knee joint

### DETAILED DESCRIPTION OF THE PREFERRED EMBODIMENTS

The present invention provides an implantable joint prosthesis configured to resurface a natural articular joint, tools, and methods for implantation. The prosthesis may include a first implant component and a second implant component. The first implant component may be implantable on a first bone and may have a first bearing surface. The second implant component may be implantable on a second bone and have a second bearing surface that corresponds to the first bearing surface.

Each bearing surface may include a flattened section such that when the bearing surfaces are placed in cooperation with one another in a preferred orientation, the flattened sections are aligned. Alternatively, the bearing surfaces may have an asymmetric configuration, with non-congruent surfaces working cooperatively. The implant corrects joint deformity by providing new articulating surfaces. The articulating surfaces restore a low coefficient of friction as well as improved fatigue, wear and loading characteristics for the joint.

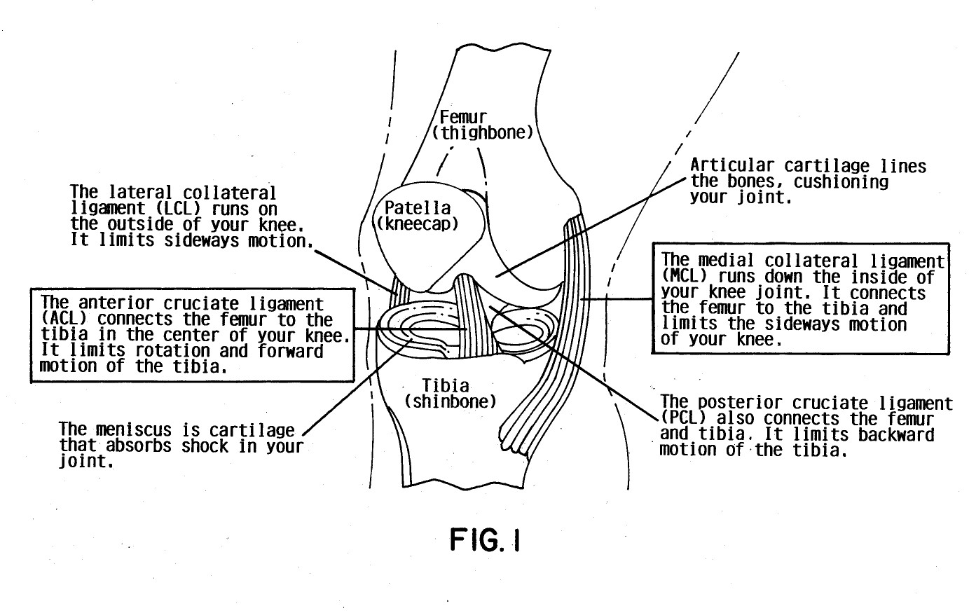

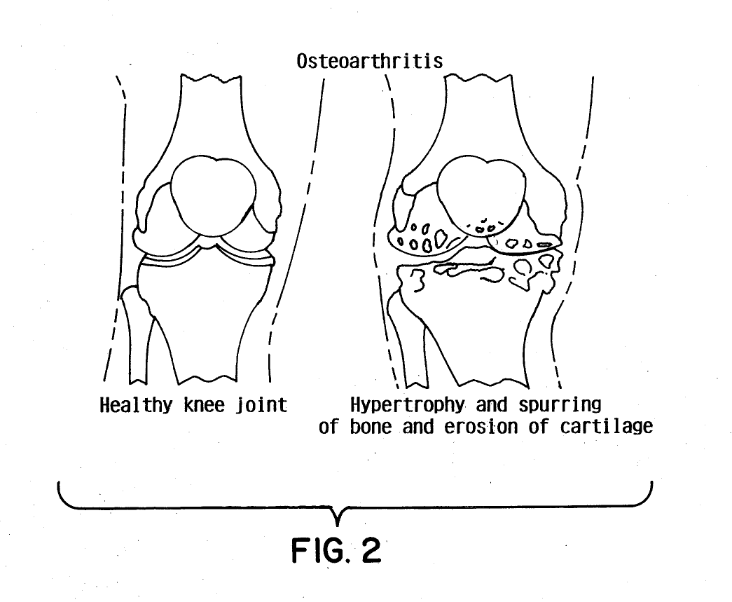

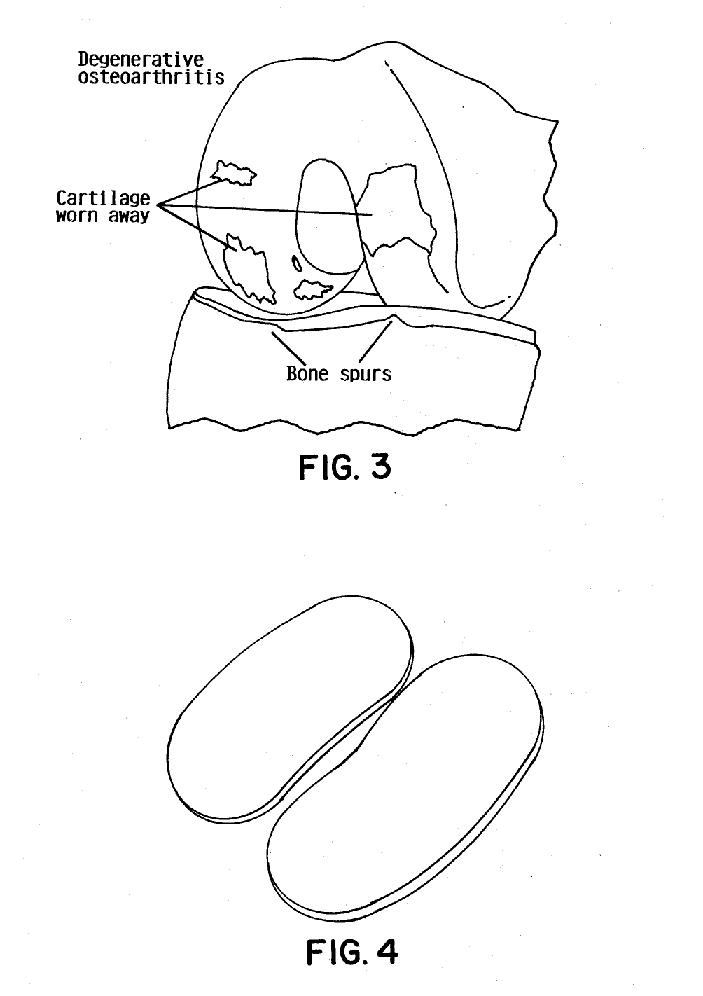

The structure of the human knee is illustrated in FIG. 1. FIG. 2 illustrates a comparison of the healthy knee cartilage and meniscus versus a severely osteoarthritic knee cartilage and meniscus. The severely osteoarthritic knee illustrated would likely require total knee replacement surgery.

Since total knee implants typically last for only 10-15 years, a patient generally cannot receive such an implant until they are at least 75 years of age. Since pain will be experienced by the patient long before the knee becomes as severely arthritic, as illustrated in FIG. 2, the present invention serves to reduce the pain level in the knee(s) of the patient until they reach an appropriate age for partial or total knee replacement surgery.

Referring to FIGS. 4-12, preferred embodiments of the invention are illustrated. The components of the implantable prosthesis are configured to be kidney-shaped, round, oval or c-shaped discs to provide resurfacing of the articular cartilage and/or menisci of corresponding mating surfaces existing in the knee joint. The selection of these component shapes are directed towards accommodating the passage of the anterior cruciate ligament through the knee joint space.

The inner and outer sides of the knee joint are referred to as lateral and medial. Lateral meaning the outside of the knee along the side of the body, and medial meaning the inside of the knee closest to the centerline of the body. One implant component resurfaces the lateral femoral condyle and the corresponding mating implant component resurfaces the lateral tibial plateau.

Likewise, the medial femoral condyle and medial tibial plateau would be resurfaced in a similar fashion. The surface contouring and flexibility of the components enable them to function together as did the native meniscus and the articular surfaces lying between the femoral condyle and tibial plateau prior to developing OA.

FIGS. 4-9 indicate only one-half of the two resurfacing components of the lateral tibial/meniscal plateau and only one-half of the two components of the medial tibial/meniscal plateau (medial being the larger of the two). The two lateral and medial femoral halves (not shown) of the conformable implant components are shaped to match the contour of the femoral condyle surface and articulate smoothly with the corresponding lateral and medial tibial/meniscal components respectively.

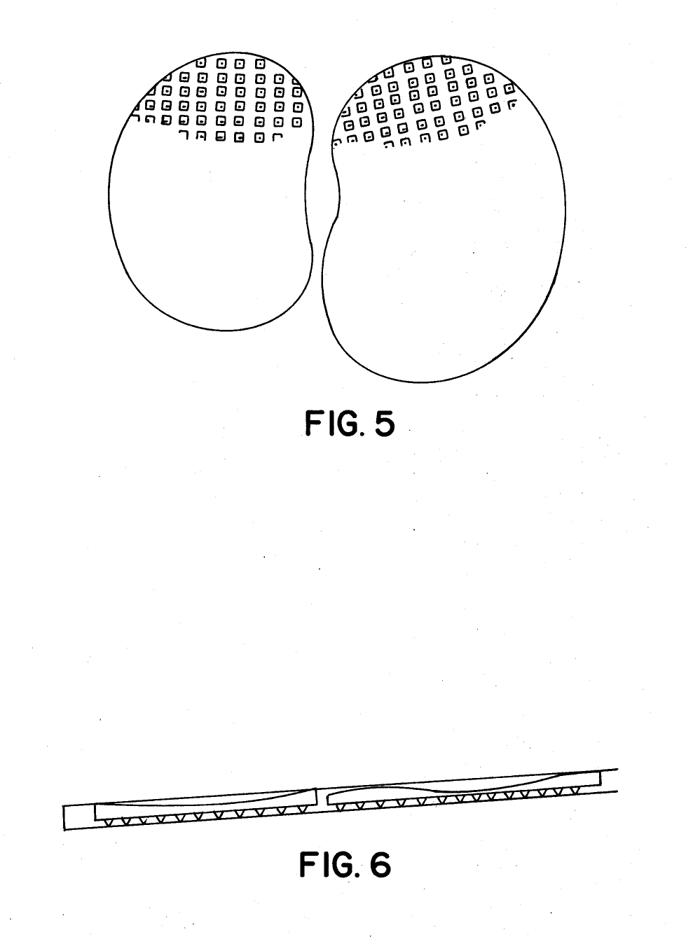

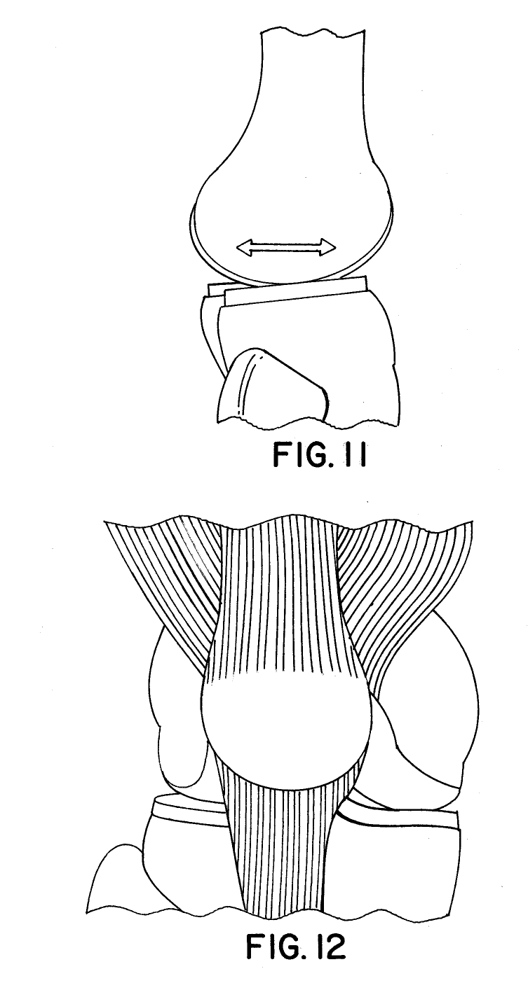

FIGS. 5 and 6 indicate mechanical engagement means in the form of teeth or spikes on the reverse side of the implant where it will be placed against the articulating cartilage. FIGS. 11 and 12 illustrate how all four components of the knee implant would appear in the joint if both lateral and medial aspects were resurfaced.

A patient with an osteoarthritic knee may need only one side of the knee joint resurfaced. The medial side of the osteoarthritic knee tends to deteriorate faster than the lateral side. The preferred embodiments of the invention may be implanted on one or both sides of the knee joint. Although the peripheral outline of the implants is anticipated to remain the same, variations in implant overall height may be required depending on the patient's procedural requirements.

Each of the resurfacing bodies exhibits sufficient flexibility to transition from a relatively flat state to an inserted state in which the resurfacing body substantially matches any multi-planar curvatures and concavities of the corresponding knee joint articular face in the presence of compressive forces associated with a typical, adult human knee joint.

With this construction, the system is capable of establishing a new sliding interface within the knee joint via articulating surfaces of the resurfacing bodies, thereby eliminating the pain-causing, bone-on-bone articular interface associated with the natural anatomy. Further, by conforming to the natural shape associated with the native knee joint articular faces, the system of the present disclosure can be inserted on a minimally-invasive basis, and restructuring (e.g., removal) of the natural bony interface is not required.

Once inserted, the surfaces of the conformable implants are formed to coapt and provide smooth articulation. Surface coaptation and articulation are accomplished by providing concave and convex aspects on the mating portions of each of the tibial/meniscal and femoral components.

It is imperative the implant discs are securely fastened into the knee joint while maintaining smooth articulating surface interaction during the loading and motion demands typically placed on the knee. Implant discs therefore may be attached to the articular cartilage of the tibia or femoral condyle with mechanical engagement. Mechanical engagement may include providing the surface of the two implant components in contact with the articular cartilage with teeth or spikes. The base web defines opposing major surfaces. The teeth and the base web define the overall height of the device.

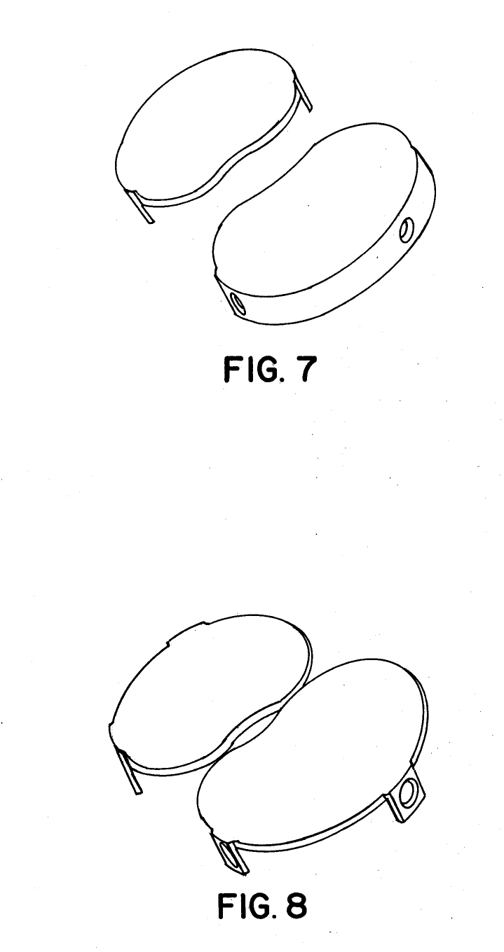

Despite the engagement of a plurality of teeth with the articular cartilage, additional mechanical engagement may also include capped edges, side tabs or flats secured with bone cement or fixation devices such as screws. FIGS. 7-10 indicate some of the various configurations anticipated to enable additional mechanical securement of implant components to the articular cartilage.

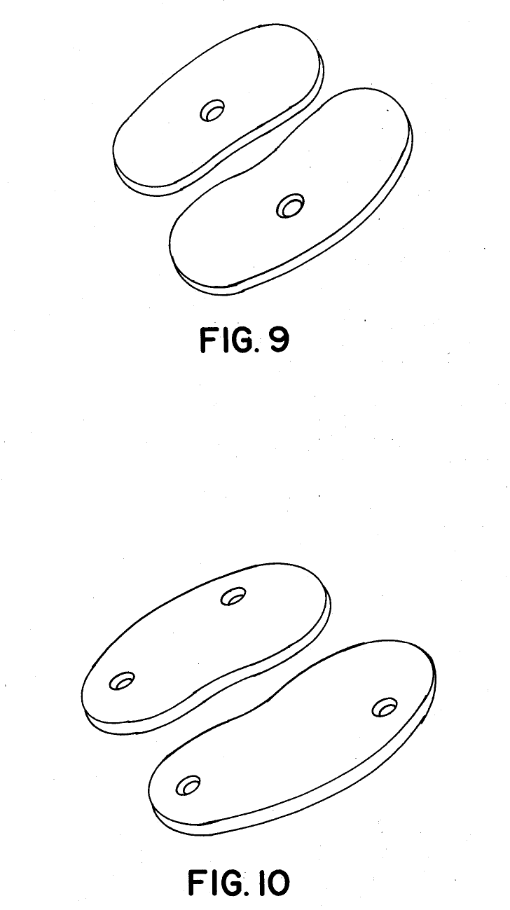

The embodiment shown in FIG. 7 includes a folded over edge creating a cap containing side holes in which screws may be inserted directly into the bone. FIG. 8 provides tabs on the side for screw fixation having eliminated the folded over edge. FIG. 9 shows top center holes and FIG. 10 shows top end holes for securement rather than side holes or tabs.

FIGS. 9 and 10 would be more feasible in the femoral condyle application as securement configurations due to the nature of the convex surface of the condyle when accessing the knee joint from the periphery of the knee in a minimally invasive or less-invasive setting. All of the embodiments would be conducive to receiving bone cement or adhesive in addition to screws to assist in fixation of the implants to the articular cartilage/bone.

Other preferred embodiments of the present invention include providing an implant configured for placement in the articulating joint between the patella and the femur (not shown). A patellar-femoral implant configuration would be a round disc rather than kidney or c-shaped.

In the preferred embodiments the overall height (or thickness) of the implant is anticipated to be consistent along its length and width.

Alternatively, the overall height may taper from one end to the other or be variable at different locations along the length and width of the implant depending on the variability existing in the diseased knee joint. The important factors are that the two-components of each pair maintain a low-friction conformable interface. In any case, the resurfacing implants are anticipated to have an overall height that ranges from 0.5-10 mm.

Yet other aspects in accordance with principles of the present disclosure relate to a kit for treating a knee joint of a patient. The kit includes a treatment system as described above (e.g., a superior resurfacing device having a superior resurfacing body, and an inferior resurfacing device having an inferior resurfacing body), along with an insertion tooling set.

The insertion tooling set includes a delivery cannula and an elongate pusher tool. The delivery cannula has a distal end and defines an internal passage that is open at the distal end. The pusher tool is sized to be slidably received within the passage. With this construction, the kit is configured to provide an insertion arrangement in which the resurfacing devices and the pusher tool are slidably received within the passage, with the resurfacing devices being stacked against one another adjacent the distal end and a distal region of the pusher tool abutting the resurfacing devices opposite the distal end of the cannula. In some embodiments, the resurfacing devices each form a notch sized to receive a finger formed by the pusher tool to achieve selective engagement therebetween.

The biocompatibility and biodurability requirements narrow the material options available for weight bearing implantable devices. Biocompatibility and biodurability are essential for permanent medical implants. The material choice cannot incite reactions such as cytotoxicity, systemic toxicity, irritation, macroscopic or allergic reactions, and muscle degeneration. The implant component materials are required to have high fatigue resistance and strength. Important material property considerations include yield strength, break strength, flexural strength, shear strength, and compressive strength.

Considering these performance requirements, the low-friction knee resurfacing implant is preferably made of medical grade polyetheretherketone (PEEK). Polyetheretherketone is an engineering thermoplastic which has been used in certain medical implant applications such as bone screws, as a component for implant medical leads and for spinal fusion cages.

It is available in pure form and also in other formulations containing additives such as carbon fiber, barium sulphate and glass fiber. Additionally the material is available as a composite comprising a PEEK matrix containing, glass and short or continuous carbon fibers for applications requiring even greater strength and rigidity. The terms “PEEK material,” or “PEEK-type material” as referenced are to include all materials of the polyaryletherketone family such as PEEK (Polyetheretherketone), PAEK (polyaryletherketone), PEK (polyetherketone), PEKK (polyetherketoneketone), PEKEKK (polyetherketoneetherketoneketone), PEEKK (polyetheretherketoneketone), and PAEEK (polyaryletheretherketone). The PEEK material selected may include the use of fillers or additives such as nanocomposites, or glass/carbon fibers.

The use of PEEK material disclosed in accordance with the preferred embodiment of the invention thus provides an implant for the knee that has a low coefficient of friction, is strong and durable, and having radiolucent properties to not interfere with imaging of the joint area. Implants made from PEEK can be repeatedly steam and gamma sterilized with no detrimental effects. These implants are inert or highly resistant to chemical attack.

Other biocompatible materials may also be used in other embodiments where the knee joint resurfacing is a temporary need, such as in sports injuries where the damaged joint slowly regenerates. One such material is a product currently under research by a company called Oxford Biomaterials. Oxford Biomaterials is developing a silk-based product called SilkBone.

SilkBone has been approved for use in humans and has mechanical load bearing properties similar to human bone with compressive forces up to 20 MPa. SilkBone is a composite of silk proteins and the natural mineral component of human bone, hydroxyapatite. An articulating joint implant having the strength and low-coefficient of friction properties of a silk-based material is additionally anticipated.

The radiolucent aspects of the PEEK material implants of the present invention may include one or more radiographic markers that are detectable by X-ray or other imaging techniques to assist in the positioning the implant during the minimally invasive surgery and to monitor its location post-implantation. Typically, these markers will be encased in predetermined locations in the implant at their periphery. Coatings that create subtle outline of the implant device during imaging may also be used, or additives such as Barium Sulphate may be included to provide some radiopacity to the implant.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A low friction resurfacing implant system comprising:
a first implant component having a first bearing surface; and
a second implant component having a second bearing surface, wherein the first implant component and the second implant component are each fabricated from a durable material that possesses a low coefficient of friction.

2. The low friction resurfacing implant system of claim 1, wherein the first bearing surface and the second bearing surface each comprise a flattened section.

3. The low friction resurfacing implant system of claim 1, wherein the first implant component and the second implant component have a similar shape but are substantially mirror images of each other.

4. The low friction resurfacing implant system of claim 1, wherein the first implant component and the second implant component are both shaped to accommodate passage of an anterior cruciate ligament through the knee joint.

5. The low friction resurfacing implant system of claim 1, wherein the first implant component is shaped to at least partially conform to a shape of a later femoral condyle, lateral tibial plateau, a medial femoral condyle and a medial tibial plateau.

6. The low friction resurfacing implant system of claim 1, wherein the implant is fabricated from PEEK (Polyetheretherketone), PAEK (polyaryletherketone), PEK (polyetherketone), PEKK (polyetherketoneketone), PEKEKK (polyetherketoneetherketoneketone), PEEKK (polyetheretherketoneketone), PAEEK (polyaryletheretherketone) or combinations thereof.

7. The low friction resurfacing implant of claim 2, and further comprising at least one additive selected from the group consisting of fillers, additives, nanocomposites, glass and carbon fibers.

8. A method a resurfacing a joint with an implant, wherein the method comprises:
providing a low friction resurfacing implant system comprising a first implant component and a second implant component, wherein the first implant component has a first bearing surface, wherein the second implant component has a second bearing surface and wherein the first implant component and the second implant component are each fabricated from a durable material that possesses a low coefficient of friction;

9. The method of claim 8, and further comprising fabricating the first bearing surface and the second bearing surface with a flattened section.

10. The method of claim 8, and further comprising forming the first implant component and the second implant component with a similar shape but in substantially mirror images of each other.

11. The method of claim 8, and further comprising forming the first implant component and the second implant component with a shape to accommodate passage of an anterior cruciate ligament through the knee joint.

12. The method of claim 8, and further comprising forming the first implant component with a shape to at least partially conform to a shape of a later femoral condyle, lateral tibial plateau, a medial femoral condyle and a medial tibial plateau.

13. The method of claim 8, and further comprising fabricating the implant from PEEK (Polyetheretherketone), PAEK (polyaryletherketone), PEK (polyetherketone), PEKK (polyetherketoneketone), PEKEKK (polyetherketoneetherketoneketone), PEEKK (polyetheretherketoneketone), PAEEK (polyaryletheretherketone) or combinations thereof.

14. The method of claim 8, and further comprising forming the implant with at least one additive selected from the group consisting of fillers, additives, nanocomposites, glass and carbon fibers.

15. The method of claim 8, wherein the implant is conformable to the bone that the implant is placed adjacent to while not translating therethrough irregularities created by the degenerated portions of the bone.

16. The method of claim 8, and further comprising fabricating the implant from silk proteins and hydroxyapatite.

17. The method of claim 8, and further comprising securing the implant with a fastening device selected from the group consisting of spikes, teeth, screws, tabs, flats folds, adhesives or combinations thereof.

18. The method of claim 8, and further comprising forming the implant with a folded over edge, wherein the folded over edge comprises at least one aperture formed therein.

19. The method of claim 8, and further comprising forming at least one aperture in the implant.

20. An implant insertion system comprising:
a delivery cannula having an internal passage extending at least partially therethrough;
an elongate pusher tool capable of slidably engaging the delivery cannula through the internal passage; and
a low friction resurfacing implant system comprising:
a first implant component having a first bearing surface; and
a second implant component having a second bearing surface, wherein the first implant component and the second implant component are each fabricated from a durable material that possesses a low coefficient of friction, wherein the first implant component and the second implant component have a height and a width that are less than a height and a width of the internal passage.

21. The implant insertion system of claim 20, wherein the first implant component and the second implant component are placed in a stacked configuration with the first bearing surface adjacent the second bearing surface.

22. The implant insertion system of claim 20, wherein at least one of the first implant component and the second implant component comprises an engagement mechanism that is capable of engaging the elongate pusher tool to retain the at least one of the first implant component and the second implant component in a desired relationship with respect to the elongate pusher tool.
